(19) United States
(12) Patent Application Publication
(10) Pub. No.: US 2007/0254963 A1
(43) Pub. Date: Nov. 1, 2007

(54) Ozonidzed Pharmaceutical Composition and Method

(75) Inventors: Haydee Alba Stenti De Pirillo, Buenos Aires (AR); Claudia Fernanda Pirillo, Buenos Aires (AR); Jose Maria Pastoriza, Buenos Aires (AR); Carlos Alberto Pastoriza, Buenos Aires (AR)

Correspondence Address: NORRIS, MCLAUGHLIN & MARCUS, P.A., 875 THIRD AVE, 18TH FLOOR, NEW YORK, NY, 10022, US

(73) Assignees: Haydee Alba STENTI DE PIRILLO, Buenos Aires (AR); Claudia Fernanda PIRILLO, Buenos Aires (AR); Jose Maria PASTORIZA, Buenos Aires (AR)

(21) Appl. No.: 11/568,155

(22) PCT Filed: Apr. 20, 2005
(86) PCT No.: PCT/US05/13223

§ 371 Date: Jul. 10, 2007

(30) Foreign Application Priority Data
Apr. 20, 2004 (AR) P 040101321

(51) Int. Cl. A61K 31/10; A01N 41/10; A61P 19/00; A61P 21/00; A61P 27/02; A61P 19/02; A01P 7/04

(52) U.S. Cl. 514/708

(57) ABSTRACT

A pharmaceutical composition to be externally applied as a topical preparation for treating several diseases, the composition comprising ozonized oils with MSM and dimethylsulfoxide (DMSO) for enhancing penetration of the active principles and permitting the treating of internal and external diseases.

### BACKGROUND OF THE INVENTION

1. Field of the Invention

The present invention relates to a pharmacological composition for topical use in the treatment of a variety of diseases, either internal or external diseases, the composition being preferably employed for treating a patient, such as a human being or animal, with ozone, and more particularly the invention relates to a composition comprising dimethylsulfoxide (DMSO), Methyl Sulfonil Methane (MSM) and a pharmaceutically acceptable vehicle, such as an ozonized vegetal oil, for extramural application, in the treatment and prevention of several diseases such as infections and infestations of several ethiology, arthritis, muscular affections, wounds and several affections in the corneal tissue. The invention also provides methods for obtaining the inventive composition by ozonizing at least one of the composition components.

2. Description of the Prior Art

It is well known to subject a patient to an ozone-based treatment for treating several diseases. The properties of the Ozone are well know in the medical field as well as it is also know the ozonization of the vehicles such as the vegetal oil, for improving the application of the ozone. The interaction of the ozone and unsaturated molecules of the vegetal oil produces the formation of a mixture of chemical compounds such as ozonides, aldehydes and peroxides. The ozonides and peroxides have germicidal activity what is very useful in the medical field.

DMSO as a therapeutic principle has been introduced in 1963 by Stanley Jacobs of the Medical School of the University of Oregon. The DMSO is a strong scavenger of free radicals, that is, when a tissue is damaged free radicals are produced which are compounds of high reactivity and capable of producing in-chain reactions that lead to more damages in the cells. The DMSO traps these free radicals as well as it breaks and stops the reaction thus preventing more damages and protecting the integrity of the cells and tissues to enhance cicatrization. Another important property of the DMSO is its synergic activity with other therapeutic components. It has been demonstrated that the activity of a standard antiviral combined with DMSO was more powerful than the components by separate in the treating of viral infections in cats (Annals of the NY Academy of Science, 1967, Vol. 141).

Dozens of primary pharmacological actions have been described for the DMSO, such as the activity on the cellular permeability, the connective tissue, inflammations, analgesia, bacteria proliferation, synergism or antagonism with other drugs, inhibition of cholinesterasic activity, diuresis, vasodilatation, cellular differentiation and functioning, antagonism in platelet aggregation, etc.

Several articles disclose the benefits of DMSO in the treatment of diseases such as intersticial cystitis, scleroderma, lupus, rheumatoid arthritis, ulcerative colitis, chronic obstructive pulmonary disease, diabetic ulceration, scar, burns, etc. which are only some examples.

The properties of the ozone and a composition comprising ozonized oils and DMSO are already disclosed in the U.S. patent application Ser. No. 10/162,284 to part of the inventors of the present application.

On the other hand the MSM is a natural sulfur source with the sulfur being a critical mineral for the normal function and structure of the human body. Sulfur is recommended in the diet of the human beings and animals and is the oxidized and primary metabolite of the DMSO and it seems that have many of the DMSO therapeutic properties. The MSM pertains to a family of compounds present in the food for terrestrial and sea life and contains sulfur as stable residue of a series of MSM compounds providing bio-available sulfur up to the 85% of the living organisms. These compounds are the few natural sources of sulfur in the earth and the MSM is in the nature such as in small proportions in fruit, vegetables, crops and beverages, such as milk that is the most abundant source of edible sulfur. Sulfur is crude material for the protein and connective tissue forming the muscles, for the enzymes conducting numerous chemical reactions and for powerful natural compounds that protect the human beings against toxicity and oxidative stress.

MSM is responsible for the flexible linking between the cells including the cells forming the skin. MSM also actuates to block undesired chemicals and crossing linking of collagen associated to a hard and aged skin. MSM improves flexibility of tissues and stimulates the repairing of damaged skin. According to tests carried out in animals, those administrated with MSM had their wounds rapidly healed. If the human body lacks MSM the new generated cells are hard and the skin has wrinkles and keloid cicatricial tissue.

In addition, the nails and hair is stronger with the provision of MSM because the cistein aminoacid that contains sulfur is present in the keratin which is the main protein in the nails and hair. The 98% of the nails is keratin. In other field, while the MSM seems to not eliminate entirely the allergic responses the patients have shown a relieve in allergic symptoms when administered with MSM.

Other studies have demonstrated that MSM has surprising anti-parasitic activity against giardia, trichomona, nematodes and other intestinal worms. The MSM competes for the receptor sites in the mucus membrane. Since the parasites can not adhere to the mucus these are rejected out from the human body, thus the MSM has a immunomodulator effect.

The MSM provides flexibility and permeability to most of the tissues. The wrong operation of the tissues along the digestive tract leads to a gastrointestinal disease such as inflammation of mucus membranes, diarrhea, wambles, hiper-acidity, defecation blocking, etc. These symptoms are alleviated by administering oral complements of MSM. It is demonstrated that some patients affected with acidity and that were receiving anti-acids preferred the use of MSM upon the better alleviation without side effects.

One of the most important applications of the organic sulfur, MSM, is the alleviation of pain associated to systemic inflammatory diseases. People with arthritis reported substantial and persistent alleviation while administered with MSM. The beneficial effect is in part due to the MSM capacity to keep the cellular flow with the damaging substances, such as lactic acid, being rejected while the nutrients are permitted to enter. MSM has a marked capacity to reduce or completely eliminate the muscular pain and cramps in the legs. The MSM is a natural medicine against most of the inflammatory muscular and skeleton problems affecting tendons and ligaments. Most of these problems are caused by repetitive and difficult movements related to job and sports.

Finally, as to the properties of the ozone, and as disclosed in U.S. Ser. No. 10/162,284, some therapeutic properties of the ozone may be generally listed as follows:

1. High germicidal power, namely bactericidal, fungicidal, viricidal and anti-parasitic.

2. Ozone improves the rheologic properties of blood and blood circulation through the capillaries.

3. Ozone increases erythrocytes' oxygen absorption capacity as well as oxygen transfer to tissues, thus improving oxygenation.

4. Ozone boosts oxygen metabolism processes through stimulation of several biochemical cycles.

5. Ozone modulates biological oxidative stress by activating the antioxidant-defense enzyme system.

6. Ozone provides Immuno-modulatory and immuno-restorative effects.

7. Ozone provides modulatory effect of biological response.

8. Ozone provides growth stimulation of granulation tissue and epithelization. Cicatrizant action.

9. Ozone revitalizes the epithelial tissue.

Today, ozone-therapy is employed medical treatments by using generally aggressive, invasive and complex techniques. Some of the treating techniques are the following:

Auto-hemotherapy: the patient must be hospitalized and intervened in a surgical room.

Injections: the ozone is injected by intramuscular or subcutaneous ways, however these injections provokes strong muscular pains in the patient because of the gaseous nature of the ozone entering into the muscular tissues.

Inter-disc injections: this consists of the injection of the ozone in the discs of the vertebral spine for treating disc hernias, however, this treatment must be excessively delicate and the needle must be guided towards and into the vertebrae disc by using computerized tomography.

Rectal administration: the ozone in gaseous form is injected via rectal, however the retention of the gas is very uncomfortable and difficult, particularly when treating animals and children.

While the composition of the above mentioned U.S. Patent Application has demonstrated very good results in the tests with several patients it has been found that the composition and the methods for obtaining the same may be remarkably improved.

### SUMMARY OF THE INVENTION

It is therefore one object of the present invention to provide a new composition and methods for obtaining the same, wherein the composition is effective for treating patients in need of the composition, with the composition combining DMSO, MSM and vegetal oils, with the capacity of enhancing penetration of the active principles in topical applications for internal or external diseases.

It is still another object of the present invention to provide a pharmaceutical composition for use in topical application to treat diseases in human beings and animals, wherein the composition comprises dimethylsulfoxide (DMSO), methyl sulfonyl methane (MSM), a pharmaceutical acceptable vehicle and products resulting from the ozonization of at least the vehicle.

It is a further object of the present invention to provide a pharmaceutical composition for use in topical application to treat diseases in human beings and animals, wherein the composition is an heterogeneous composition having an upper phase and a lower phase, wherein the lower phase comprises DMSO and MSM, and the upper phase comprises vegetal oil and products resulting from the ozonization of the vegetal oil, with the upper phase comprising between about 50% to about 90% of the total volume of the composition and the lower phase comprising between about 10% to about 50% of the total volume of the composition.

It is a further object of the present invention to provide a pharmaceutical composition for use in topical application to treat diseases in human beings and animals, wherein the composition is an heterogeneous composition having an upper phase and a lower phase, wherein the upper phase contains vegetal oil and the products resulting from the ozonization of the oil and the lower phase contains DMSO and MSM in an amount between about 1% w/w and about 10% w/w.

It is a further object of the present invention to provide a pharmaceutical composition for use in topical application to treat diseases in human beings and animals, wherein the composition comprises DMSO, MSM and ozonized oils.

It is another object of the present invention to provide a method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:

i. mixing pharmaceutical acceptable amount of the vegetal oil and a pharmaceutical acceptable amount of dimethylsulfoxide (DMSO) to obtain a mixture, and

ii. ozonizing the mixture obtained in step i until obtaining a pharmaceutical acceptable amount of Methyl Sulfonil Methane (MSM).

It is still another object of the present invention to provide a method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:

i. ozonizing a pharmaceutical acceptable amount of dimethylsulfoxide (DMSO) to obtain a first product comprising a pharmaceutical acceptable amount of Methyl Sulfonyl Methane (MSM);

ii. ozonizing a pharmaceutical acceptable amount of the vegetal oil to obtain a second product resulting from the ozonization, and

iii. mixing the first and second products.

It is even another object of the present invention to provide a method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:

i. providing a pharmaceutical acceptable amount of dimethylsulfoxide (DMSO);

ii. providing a pharmaceutical acceptable amount of Methyl Sulfonyl Methane (MSM);

iii. providing a pharmaceutical acceptable amount of vegetal oil;

iv. ozonizing the vegetal oil, and

v. mixing the DMSO, MSM and ozonized vegetal oil.

The above and other objects, features and advantages of this invention will be better understood when taken in connection with the following description.

### DESCRIPTION OF THE PREFERRED EMBODIMENTS

### Definitions

While the following list is not restrictive, the same indicates some preferable parts of an animal body and diseases or disorders that may be treated by the present invention.

Coronary Band: direct trauma and contusion, penetration by foreign bodies and infection, laceration, avulsion, displacement, dermopathies (mycotic, chemical, allergic, parasitic, neoplastic), tearing away.

Hoof Wall: fracture in any of its locations (bars included), submural infection (foreign bodies), tearing away of wall, loss of wall or avulsion, wall anomalies (localized lack of growth, formation of hoof marks, wearing away).

Sole: subsolar contusion, subsolar penetration and infection, sole laceration or loss, penetration via the distal phalanx, subsolar hematoma (seroma), excessively thin, weak or flat sole.

Laminar Tissue: founder, keratoma, infection, submural hematoma or tearing, metastasis, abnormal cornification resulting from chronic tearing away of the wall.

Frog: intertrigo, cancer, penetration and infection, loss resulting from avulsion, contusion or atrophy.

Heel Bulbs: direct trauma and contusion, laceration, avulsion, dermopathies (mycotic, chemical, parasitic, neoplastic).

Distal Sesamoid: Navicular disease (syndrome), infection (osteomyelitis), diseases of the podotrochlear bursa (traumatic, infectious, idiopathic).

Distal Phalanx (Medial and Lateral) Cartilages: ossification, infection or aseptic necrosis (collateral cartilage infection), fracture of calcified cartilages, trauma (contusion).

Veterinary Pathologies:

Pathologies in corneous tissues: hematoma, dehydration, abscess, thinness, injuries and wounds.

Pathologies in osseous tissues: acute arthritis, chronic arthritis (naviculitis, navicularthritis), degenerative arthritis, ceruse arthritis, arthrosis, hemorrhagic arthritis, articular and osseous injuries and wounds.

Pathologies in muscular tissues: acute myositis, chronic myositis, tearing, hematoma, edema, fibrosis, injuries and wounds.

Pathologies in tendinous tissues: inflammation, edema, hemorrhages, tearing, injuries and wounds.

Pathologies in mammary tissues: inflammation, infection, edema, hemorrhages, injuries and wounds.

Pathologies in the circulatory system: phlebitis and inflammation.

Soft tare: inflammation, edema, hemorrhages, bursitis, injuries and wounds.

Tumoral malformations: cystic follicular granuloma and sarcoid.

Pathologies in dermal tissues: dermatitis, folliculitis, abscess, infection.

Human Pathologies:

Podiatry: callosity, plantar callus, foot muscular pain.

Traumatology: arthritis, arthrosis, muscular pains, tendon affections, muscular tearing, rheumatism.

Phlebology: circulatory disorders, edema, bloal.

While most of the references will be made to the use of the present composition to the application in the treatment of diseases in horses, many tests have been carried out with success in the treatment of human diseases such as in podiatry for skin affections, ulcers, lesions, fragile nails, arthritic and muscular pains, etc.

Now referring in detail to the invention the same provides a pharmacological or pharmaceutical composition in any topical desired preparation for treating several diseases by means of ozone or ozonized oxygen, internal or external diseases. More particularly, the composition is the combination of ozonized vegetal oils, DMSO and MSM that permits the penetration of the transformation products, namely the products resulting from the ozonization of the vegetal oils, through the skin and dermal layers for treating internal diseases for example, applying the composition in a zone of the body close to the internal affected organ.

As mentioned above, a topical composition is disclosed in the U.S. Ser. No. 10/162,2840 containing DMSO and vegetal oils however, the inventors have found that this composition can be improved by the addition of MSM in a predetermined concentration which MSM not only provides its own properties to the composition but unexpected enhanced properties of the composition and production conditions have been found. The inventive composition provides:

a. Control and monitoring of the conditions of the ozonization reaction in order to stop reaction upon reaching the amount of between about 1% w/w and 10% w/w of MSM when the method of obtaining the composition comprises the steps of mixing of DMSO with vegetal oil to obtain a mixture and ozonizing the mixture.

b. Facility in the industrial processes for producing the inventive composition by permitting to ozonize only the vegetal oil and, as a consequence of this, to increase the productivity of the reactors when the method of obtaining the composition comprises the steps of ozonizing the vegetal oil and adding DMSO and MSM to the ozonized oil up to reaching a concentration of between 1% and 10%.

The invention may be better understood with reference to the following examples which are not limitative or restrictive of the scope of protection. On the contrary, it must be clearly understood that many other embodiments, modifications and alterations equivalent to the elements of the invention may be suggested by persons skilled in the art after reading the present description, without departing from the spirit of the present invention and/or the scope of the appended claims.

### EXAMPLES

### Example 1

### Method of Preparing the Composition

According to this alternative method 25 ml vegetal oil and 25 ml DMSO were mixed into a 50:50 v/v mixture. The mixture was ozonized for 20 minutes under controlled flow and the reaction was monitored by detecting the presence of MSM in the mixture. The working conditions, flow and ozonization time were conveniently standarized and optimized taking as a reference the appearance of the MSM preferably in an amount of between 1% and 10%. The reaction was permitted to stop with the MSM in the mentioned values.

### Example 2

### Antimicrobic Activity In Vitro

This test was carried out to compare the antimicrobic activity in vitro of the inventive composition and the vegetal oil alone. The following dilutions in triplicate were prepared, that is samples (ozonized vegetal oil and fluids of the invention) in nutritive broth: 5%, 2.5%, 0.5%, 0.25%, 0.1% and 0.01% w/v. The broth was placed in tubes and each tube was inoculated with 6 10⁷ CFU (colonies forming units) of Staphylococcus aureus and incubated for 24 hrs. at 37° C. After incubation a sub-culture of each dilution was carried out in agar tripticasa-soy. Table 1 shows the obtained results.

NOTE: The tests have been carried in laboratory with multiple replicas, with the results herein shown being an average of the results obtained with the replicas.

TABLE 1

| | | | | Composition of the | | |
|---|---|---|---|---|---|---|
| Concentration | Ozonized Oil | | | Invention | | |
| % w/v | 1 | 2 | 3 | 1 | 2 | 3 |
| 0.01 | +++ | +++ | +++ | +++ | +++ | +++ |
| 0.10 | +++ | +++ | +++ | +++ | +++ | +++ |
| 0.25 | +++ | +++ | +++ | +++ | +++ | +++ |
| 0.50 | +++ | +++ | +++ | + | + | + |
| 2.50 | ++ | ++ | ++ | + | + | + |
| 5.00 | + | + | + | − | − | − |

(+++) Abundant development
(++) Medium development
(+) Light development
(−) No development

### Conclusion

The above results clearly show that the antimicrobic activity of the inventive composition (fluid) is higher than the ozonized oil employed alone.

### Example 3

### Antimycotic Activity In Vitro

This test was carried out to compare the antimycotic power of the present composition and the one of an ozonized oil.

Table 2 shows the activity of the inventive composition and a vegetal oil by separate over several species of fungus and yeast such as Penicillium, Aspergillus, Fusarium, and Candida Albicans. In each case all the microorganisms were treated with ozonized oil and the inventive composition comprising ozonized oil, DMSO and MSM.

TABLE 2

| | | Composition of the |
|---|---|---|
| Cells | Ozonized Oil | Invention |
| Penicillium | +++ | ++ |
| Aspergillus | +++ | +++ |
| Fusarium | +++ | + |
| Candida albicans | +++ | ++ |

(+++) Abundant development
(++) Medium development
(+) Light development

### Conclusion

The microorganisms treated with the inventive composition show a higher inhibition to grow as compared to those treated with the ozonized oil.

### Example 4

### Evaluation of Oxidizing Agents

The presence of oxidant agents in the present composition has been evaluated by the reaction with iodine in starch. The reaction is based in the oxidation of the iodide under the action of an oxidant agent, the ozone, resulting in free iodine having a strong blue color in a solution of starch.

When analyzing the inventive composition by means of the reactant mentioned above no blue color was observed with the potassium/starch iodide.

### Conclusion

The sample of the invention does not contain free oxidant agents, including ozone.

### Example 5

### Evaluation of Acidity of the Composition of the Invention

The acidity of four (4) samples according to Rule IRAM 5514NIO/1955 modified for heterogeneous base. The evaluated samples were the following:

DMSO alone: A DMSO sample was ozonized for 20 minutes under a controlled rate.

Vegetal oil alone: A sample of vegetal oil was ozonized at a controlled rate.

DMSO in mixture: A mixture or composition of the invention was prepared having a lower phase comprising DMSO and MSM, and an upper phase comprising vegetal oil and products resulting from the ozonization of the vegetal oil. The sample of DMSO consisted of a sample taken from the lower phase of the mixture.

Vegetal Oil in Mixture:

This sample was taken from the upper phase of a mixture prepared like the mixture for taken the above mentioned sample of DMSO in mixture.

The results are shown in Table 3.

TABLE 3

| | | Oil in | DMSO in | |
|---|---|---|---|---|
| Acidity | Oil alone | mixture | mixture | DMSO alone |
| g NaOH/100 g | 0.32 | 7.03 | 18.03 | 0.93 |
| of sample | | | | |

### Conclusion

An increased acidity is observed for the oil and the DMSO when the same are ozonized together in the mixture. This does not occur when the same are ozonized by separate. Therefore the DMSO could enhance the appearance of components giving more acidity to the mixture.

### Example 6

### Action of the Ozone over the DMSO and over the Vegetal Oil/DMSO Mixture

The action of the Ozone over the DMSO was evaluated by high-resolution mass chromatography—Mass Spectometry (GC/MS). The starting sample was no-ozonized DMSO and the results are shown in Table 4.

TABLE 4

| Component | g./100 g of sample |
|---|---|
| DMSO | 99.96 |
| MSM | 0.04 |

Afterwards, a portion of the sample was subjected to a ozonization process for 20 minutes under controlled rate and the results were analyzed by GS/MS (DMSO ozonized alone).

The lower phase of the inventive composition, represented by the DMSO (DMSO in mixture), as disclosed in Example 5, was also analyzed. The results are shown in Table 5.

TABLE 5

| | | DMSO ozonized in |
|---|---|---|
| | DMSO ozonized alone | mixture |
| Component | g./100 g sample | g./100 g sample |
| DMSO | 94.8 | 94.3-98.3 |
| MSM | 5.2 | 1.7-5.7 |

### Conclusions

The Ozone transform the DMSO into MSM and this is very useful and convenient because the MSM not only provides to the composition the own properties of the MSM but also the MSM permits to have a control over the ozonization process, as explained above, since the appearance and amount thereof provides the indication when the process must be interrupted.

### Example 7

### Evaluation of the Transformation Products Resulting from the Transformation of the Vegetal Oil

A sample of ozonized vegetal oil and a sample of ozonized vegetal oil and DMSO have been evaluated by gaseous chromatography. The components detected by this technique are only the organic volatile substances.

An analyzed sample of non-ozonized vegetal oil comprised as main component linoleic acid and, as minor components, palmitic, stearic and oleic acid. Then, the sample was ozonized for 20 minutes at a controlled rate and the analysis results are shown in Table 6.

TABLE 6

| Component | g./100 g sample |
|---|---|
| Hexanal | 0.3 |
| Nonanal + Octanoic Acid | 1.0 |
| 9-Oxo Nonanoic Acid | 2.6 |
| Palmitic Acid | 6.0 |
| Linoleic Acid | 90.1 |

The upper phase of the inventive composition, represented by the vegetal oil, as disclosed in Example 5, was also analyzed. The results are shown in Table 7.

TABLE 7

| Component | g./100 g sample |
|---|---|
| Hexanal | 0.1 |
| Nonanal + Octanoic Acid | 0.3 |
| 9-Oxo Nonanoic Acid | 0.8 |
| Palmitic Acid | 6.0-16.1 |
| Linoleic Acid | 82.9-92.8 |

### Conclusions

The ozonization process on the vegetal oil produces the appearing of transformation products coming from the unsaturated acids, resulting in compounds having a lower molecular weight, such as aldehydes, shorter-chain fatty acids and ozonides. The appearance of these compounds is due to the action of the ozone over the double-link.

The quantitative differences may be due to, among other causes, the presence of DMSO during the ozonization process. The quantitative differences in the results from the analysis of the replicas of the inventive composition are shown in Table 7 and are shown as a rate of values. This may be due, for instance to: it is a non-homogeneous composition having two phases; the probable evaporation of volatile compounds during the reaction conditions, or an unequal distribution of the components in both phases of the composition.

### Example 8

### Treatment of Parasitical Nodules in Equines

Tinea is a contagious parasitic disease that affects not only to human but also to animals and its etiology varies depending of the affected species. The signs and symptoms include nodules in the skin, loosing of hair in the affected area, inflammation, pruritus, joining of the nodules in the surface, loosing of skin in the nodules, etc.

The prior art treatment consisted of repose, application of sodium hypochlorite in several concentrations and treatment with several antimycotic.

According to the invention, ten (10) equines affected by tinea have been treated with topical administration of the inventive composition onto the nodules for about 16 days. In three (3) of these animals the repose period was not complete because they were in the middle of a competing season. At the day 18 counted from the diagnosis the horses were permitted to return to normal activities in healthy conditions.

It is important to remark that no other medication was administered to the animals, neither local nor systemic medicines. Therefore, it is to be remarked that no antimycotics were necessary.

### Example 9

### Treatment of Wounds

The treatment of open wounds in animals it complex because the animal tend to bite the affected area due to the discomfort in that zone, thus the wound is re-opened with the consequents risks of infection and infestation generated not only by the saliva but by fly larvae.

According to the invention twelve (12) horses having open wounds of about 8 to 12 cm. were treated with the inventive fluid or composition. The treatment comprised the cleaning of the wounds and the local application of the composition of the invention and the suturing of the wounds. The treatments were done daily for a period of 12 to 21 days. No further local medication was administered, neither in local form nor in systemic form. While the treatments were carried out in summer time no fly larvae were observed in the wounds.

One of the animals had a wound that was open for about ten hours without suture. This animal used to bite the wound and the wound was re-opened in the middle of the treatment.

### Example 10

### Treatment of Claudication

The claudication is a common affection particularly in equines for sportive activities. The affection comprises an alteration in the natural capacity of doing movements either in humans and animals.

According to this Example 18 equines affected with claudication in forearms and rear legs have been treated by massaging the affected areas with the inventive composition and repose. The animals were healthy and permitted to return to activities between the day 9 and day 12 from the diagnosis. No further local medication was administered, neither in local form nor in systemic form. A high analgesic and anti-inflammatory power of the inventive composition has been observed.

### Example 11

### Treatment of Acute Arthritis in Equines

Seven (7) equines were selected from a group for sportive activities, with all of them being affected by arthritis in some of their limbs. Among other symptoms they were affected by pain upon touching, inflammation and difficulties in doing some movements. All the horses were treated with daily topical applications of the composition of the invention with massage in the affected zone for a period of ten (12) days. After five (5) days from the beginning of the treatment the horses started to look better with pain reduction and the inflammation in the affected areas was reduced. After eleven (11) days from the beginning of the treatment the horses returned again to the sport training.

In this Example, the capacity of the inventive composition to treat inner affections by topically applying the composition in the body closer to the affected inner organ is demonstrated.

### Example 12

### Treatment of Horny Tissue, Fractures

These lesions in the hooves of equines are very common particularly fractures in horses that compete in jumping sports. These lesions however are not restricted to this group but they appear when the animal is wounded with sharp objects that punctures a hoof and produces a fracture. The lameness is an indication that a hoof has a deep fracture and this is accompanied by hemorrhages.

According to this Example of the invention four (4) equines having hoof fractures were treated with the inventive composition. The composition was topically applied onto the affected hooves and close areas thereof. At about the four days of treatment a reduction of pain was observed. As from about day 9 the affected hooves started to show a good evolution and growth.

### Example 13

### Treatment of Horny Tissue. Fragile and Weak Hooves

Twenty two (22) equines affected by fragile hooves were treated with daily topical applications of the composition of the invention. Remarked improvements were observed and the horses returned to the training activities after three (3) and four (4) months as from the starting of the treatment. The animals did not received any other medical treatment and the first improvements were observed at a week from the starting of the treatment.

While preferred embodiments of the present invention have been illustrated and described, it will be obvious to those skilled in the art that various changes and modifications may be made therein without departing from the scope of the invention as defined in the appended claims.

We claim:

1. Pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle.

2. The composition of claim 1, wherein the vehicle is selected from the group comprising vegetal oil, hydrocarbon derivatives, vaseline, paraffin, wax and lanoline.

3. The composition of claim 2, wherein the vegetal oil is selected from the group comprising soy oil, corn oil, sunflower oil, olive oil, jojoba oil and peanut oil.

4. The composition of claim 1, wherein the products resulting from the transformation of the vehicle are selected from the group comprising aldehydes and fatty acids.

5. The composition of claim 1, wherein the composition has infection and infestation preventing activity, pain mitigating activity, cicatrization activity, cheloid inhibiting activity and insects repellent activity.

6. The composition of claim 1, comprising an heterogeneous composition having an upper phase and a lower phase, wherein the lower phase comprises DMSO and MSM, and the upper phase comprises vegetal oil and products resulting from the ozonization of the vegetal oil.

7. The composition of claim 6, wherein the upper phase comprises between about 50% to about 90% of the total volume of the composition and the lower phase comprises between about 10% to about 50% of the total volume of the composition.

8. The composition of claim 6, wherein the lower phase contains DMSO and between about 1% w/w and about 10% w/w of MSM.

9. The composition of claim 1, comprising a preparation selected from the group consisting of ointment, unguent, salve, cream, emulsion, suspension, suppository, ovules and solution.

10. The composition of claim 1, wherein the composition is presented in an application carrier selected from the group comprising cloth, pad, paper, fabric, wool, controlled releasing support, spray, aerosol and brush.

11. The composition of claim 1, having podiatric activity.

12. The composition of claim 1, having analgesic and anti-inflammatory activity.

13. A method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:
i. mixing pharmaceutical acceptable amount of the vegetal oil and a pharmaceutical acceptable amount of dimethylsulfoxide (DMSO) to obtain a mixture, and
ii. ozonizing the mixture obtained in step i until obtaining a pharmaceutical acceptable amount of Methyl Sulfonil Methane (MSM).

14. The method of claim 13, wherein the amount of MSM is between about 1% w/w and about 10% w/w.

15. The method of claim 13, wherein the ozonizing step provides products resulting from the transformation of unsaturated acids of the vegetal oil, the amount of these transformation products is between about 0.5% w/w and about 20% w/w.

16. The method of claim 13, wherein the ozonizing step is carried out under conditions necessary to obtain an amount between about 1% w/w and 10% w/w of MSM.

17. A method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:
i. ozonizing a pharmaceutical acceptable amount of dimethylsulfoxide (DMSO) to obtain a first product comprising a pharmaceutical acceptable amount of Methyl Sulfonyl Methane (MSM);
ii. ozonizing a pharmaceutical acceptable amount of the vegetal oil to obtain a second product resulting from the ozonization, and
iii. mixing the first and second products.

18. The method of claim 17, wherein the pharmaceutical amount of MSM is between about 1% w/w and about 10% w/w.

19. The method of claim 17, wherein the ozonization of the vegetal oil provides products resulting from the transformation of unsaturated acids of the vegetal oil, the amount of these transformation products is between about 0.5% w/w and about 20% w/w.

20. The method of claim 17, wherein the ozonizing step i) is carried out under conditions necessary to obtain an amount between about 1% w/w and 10% w/w of MSM.

21. A method for obtaining a pharmaceutical composition for use in topical application to treat diseases in human beings and animals, the composition comprising dimethylsulfoxide (DMSO), methyl sulfonyl methane, a pharmaceutical acceptable vehicle and products resulting from the ozonization of at least the vehicle, the method comprising:
i. providing a pharmaceutical acceptable amount of dimethylsulfoxide (DMSO);
ii. providing a pharmaceutical acceptable amount of Methyl Sulfonyl Methane (MSM);
iii. providing a pharmaceutical acceptable amount of vegetal oil;
iv. ozonizing the vegetal oil, and
v. mixing the DMSO, MSM and ozonized vegetal oil.

22. The method of claim 21, wherein the pharmaceutical acceptable amount of MSM is between about 1% w/w and 10% w/w.

23. The method of claim 21, wherein the ozonization of the vegetal oil provides products resulting from the transformation of unsaturated acids of the vegetal oil, the amount of these transformation products is between about 0.5% w/w and about 20% w/w.
